(19) United States
(12) Patent Application Publication
(10) Pub. No.: US 2011/0009446 A1
(43) Pub. Date: Jan. 13, 2011

(54) OLIGOMER-GUANIDINE CLASS CONJUGATES

(75) Inventors: Wen Zhang, Madison, AL (US); Stephanie Allums, Huntsville, AL (US); Jennifer Riggs-Sauthier, Huntsville, AL (US)

Correspondence Address: NEKTAR THERAPEUTICS, 201 INDUSTRIAL ROAD, SAN CARLOS, CA, 94070, US

(73) Assignee: Nektar Therapeutics, San Carolos, CA (US)

(21) Appl. No.: 12/811,374

(22) PCT Filed: Jan. 9, 2009
(86) PCT No.: PCT/US2009/000140

§ 371 Date: Sep. 22, 2010

Related U.S. Application Data
(60) Provisional application No. 61/010,832, filed on Jan. 11, 2008.

(51) Int. Cl. C07D 217/04; A61K 31/47

(52) U.S. Cl. 514/307; 546/145

(57) ABSTRACT

The invention relates to (among other things) oligomer-guanidine class conjugates and related compounds. A conjugate of the invention, when administered by any of a number of administration routes, exhibits advantages over previously administered compounds.

### CROSS REFERENCE TO RELATED APPLICATION

This application claims the benefit of priority under 35 U.S.C. §119(e) to U.S. Provisional Patent Application Ser. No. 61/010,832, filed 11 Jan. 2008, the disclosure of which is incorporated herein by reference in its entirety.

### FIELD OF THE INVENTION

This invention comprises (among other things) chemically modified guanidine class compounds that possess certain advantages over guanidine class compounds lacking the chemical modification. The chemically modified guanidine class compounds described herein relate to and/or have application(s) in (among others) the fields of drug discovery, pharmacotherapy, physiology, organic chemistry, and polymer chemistry.

### BACKGROUND OF THE INVENTION

Hypertension, commonly referred to as “high blood pressure” or HTN, is a condition in which the blood pressure is chronically elevated. While it is formally called arterial hypertension, the word “hypertension” without a qualifier usually refers to arterial hypertension. Hypertension can be classified as either essential (primary) or secondary. Essential hypertension indicates that no specific medical cause can be found to explain a patient's condition. Secondary hypertension indicates that the high blood pressure is a result of (i.e. secondary to) another condition, such as kidney disease or certain tumors (especially of the adrenal gland). Persistent hypertension is one of the risk factors for strokes, heart attacks, heart failure and arterial aneurysm, and is a leading cause of chronic renal failure. Even moderate elevation of arterial blood pressure leads to shortened life expectancy.

Hypertension may be treated using several different approaches. ACE inhibitors, angiotensin II receptor antagonists, alpha blockers, beta blockers, calcium channel blockers, rennin inhibitors, and diuretics are some of the antihypertensive drugs. Guanidine derivatives are sympathoplegic agents that apparently lower blood pressure by reducing peripheral vascular resistance, inhibiting cardiac function, and increasing venous pooling in capacitance vessels. Guanidine derivatives may exert their effect by acting on sympathetic nerve terminals and/or vasomotor center. Drugs that lower blood pressure by actions on the central nervous system tend to cause sedation, mental depression, and sleep disturbance. Drugs that act on autonomic ganglia show parasympathetic blockage. Drugs that reduce the release of norepinephrine may lead to inhibition of ejaculation, and hypotension. These drugs are metabolized by liver and therefore require higher dosing. Therefore, pharmacotherapy with antihypertensives would be improved if these side effects associated with their use could be decreased or if their pharmacokinetics could be improved. Thus, there is a large unmet need for developing novel guanidine class compounds. The improved guanidine class compounds may also show other therapeutic effects, i.e. analgesic activity.

The present invention seeks to address these and other needs in the art.

### SUMMARY OF THE INVENTION

In one or more embodiments of the invention, a compound is provided, the compound comprising a guanidine class residue covalently attached via a stable or degradable linkage to a water-soluble, non-peptidic oligomer.

Exemplary compounds of the invention include those having the following structure:

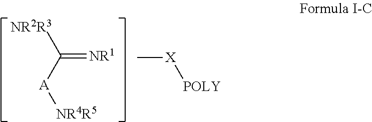

wherein:
A is selected from the group consisting of nil, lower alkylene, —CH₂NH—, and —CH₂CH₂NH—;
R¹ is selected from the group consisting of hydrogen, alkyl, cyano, and aryl;
R² and R³ are each independently selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

Exemplary compounds also include compounds with the following structure:

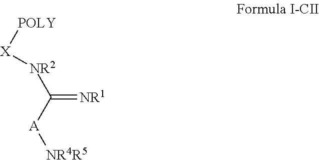

wherein:
A is selected from the group consisting of nil, lower alkylene, —CH₂NH—, and —CH₂CH₂NH—;
R¹ is selected from the group consisting of hydrogen, alkyl, cyano, and aryl;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

Exemplary compounds also include compounds with the following structure:

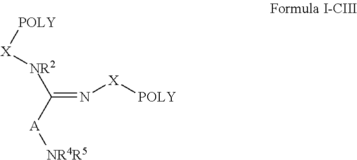

wherein:
A is selected from the group consisting of nil, lower alkylene, —CH₂NH—, and —CH₂CH₂NH—;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

The “antihypertensive residue” is a compound having a structure of a guanidine class antihypertensive compound in the guanidine class that is altered by the presence of one or more bonds, which bonds serve to attach (either directly or indirectly) one or more water-soluble, non-peptidic oligomers. Exemplary antihypertensives have a structure encompassed by at least one of the structures defined herein as Formula I:

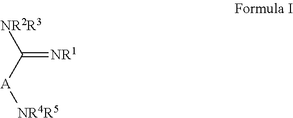

wherein:
A is selected from the group consisting of nil, lower alkylene, —CH₂NH—, and —CH₂CH₂NH—;
R¹ is selected from the group consisting of hydrogen, alkyl, cyano, and aryl;
R² and R³ are each independently selected from the group consisting of hydrogen, hydroxyl, alkyl, substituted alkyl, aryl, substituted aryl, and alkylaryl; and
R⁴ and R⁵ are each independently selected from the group consisting of hydrogen, hydroxyl, alkyl, substituted alkyl, aryl, substituted aryl, heteroaryl and alkylaryl, or R⁴, R⁵, and the nitrogen to which they are attached, together form heteroaryl or heterocycle.

In one or more embodiments of the invention, a composition is provided, the composition comprising a compound comprising a guanidine class antihypertensive residue covalently attached via a stable or degradable linkage to a water-soluble, non-peptidic oligomer, and optionally, a pharmaceutically acceptable excipient.

In one or more embodiments of the invention, a dosage form is provided, the dosage form comprising a compound comprising a guanidine class antihypertensive residue covalently attached via a stable or degradable linkage to a water-soluble, non-peptidic oligomer, wherein the compound is present in a dosage form.

In one or more embodiments of the invention, a method is provided, the method comprising covalently attaching a water-soluble, non-peptidic oligomer to a guanidine class antihypertensive.

In one or more embodiments of the invention, a method is provided, the method comprising administering a compound comprising a guanidine class antihypertensive residue covalently attached via a stable or degradable linkage to a water-soluble, non-peptidic oligomer.

These and other objects, aspects, embodiments and features of the invention will become more fully apparent to one of ordinary skill in the art when read in conjunction with the following detailed description.

### BRIEF DESCRIPTION OF THE FIGURES

FIG. 1. Mean (±SEM) percent specific binding of debrisoquine and PEG-debrisoquine to noradrenalin transporters in rat forebrain membranes.

### DETAILED DESCRIPTION OF THE INVENTION

As used in this specification, the singular forms “a,” “an,” and “the” include plural referents unless the context clearly dictates otherwise.

In describing and claiming the present invention, the following terminology will be used in accordance with the definitions described below.

“Water soluble, non-peptidic oligomer” indicates an oligomer that is at least 35% (by weight) soluble, preferably greater than 70% (by weight), and more preferably greater than 95% (by weight) soluble, in water at room temperature. Typically, an unfiltered aqueous preparation of a “water-soluble” oligomer transmits at least 75%, more preferably at least 95%, of the amount of light transmitted by the same solution after filtering. It is most preferred, however, that the water-soluble oligomer is at least 95% (by weight) soluble in water or completely soluble in water. With respect to being “non-peptidic,” an oligomer is non-peptidic when it has less than 35% (by weight) of amino acid residues.

The terms “monomer,” “monomeric subunit” and “monomeric unit” are used interchangeably herein and refer to one of the basic structural units of a polymer or oligomer. In the case of a homo-oligomer, a single repeating structural unit forms the oligomer. In the case of a co-oligomer, two or more structural units are repeated—either in a pattern or randomly—to form the oligomer. Preferred oligomers used in connection with present the invention are homo-oligomers. The water-soluble, non-peptidic oligomer typically comprises one or more monomers serially attached to form a chain of monomers. The oligomer can be formed from a single monomer type (i.e., is homo-oligomeric) or two or three monomer types (i.e., is co-oligomeric).

An “oligomer” is a molecule possessing from about 1 to about 30 monomers. Specific oligomers for use in the invention include those having a variety of geometries such as linear, branched, or forked, to be described in greater detail below.

“PEG” or “polyethylene glycol,” as used herein, is meant to encompass any water-soluble poly(ethylene oxide). Unless otherwise indicated, a “PEG oligomer” an oligoethylene glycol is one in which substantially all (preferably all) monomeric subunits are ethylene oxide subunits, though the oligomer may contain distinct end capping moieties or functional groups, e.g., for conjugation. PEG oligomers for use in the present invention will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. As stated above, for the PEG oligomers, the variable “n” ranges from about 1 to 30, preferably from about 2 to about 30, and the terminal groups and architecture of the overall PEG can vary. When PEG further comprises a functional group, A, for linking to, e.g., a small molecule drug, the functional group when covalently attached to a PEG oligomer does not result in formation of (i) an oxygen-oxygen bond (—O—O—, a peroxide linkage), or (ii) a nitrogen-oxygen bond (N—O, O—N).

The terms “end-capped” and “terminally capped” are interchangeably used herein to refer to a terminal or endpoint of a polymer having an end-capping moiety. Typically, although not necessarily, the end-capping moiety comprises a hydroxy or C₁₋₂₀ alkoxy group, more preferably a C₁₋₁₀ alkoxy group, and still more preferably a C₁₋₅ alkoxy group. Thus, examples of end-capping moieties include alkoxy (e.g., methoxy, ethoxy and benzyloxy), as well as aryl, heteroaryl, cyclo, heterocyclo, and the like. It must be remembered that the end-capping moiety may include one or more atoms of the terminal monomer in the polymer [e.g., the end-capping moiety “methoxy” in CH₃O(CH₂CH₂O)ₙ— and CH₃(OCH₂CH₂)ₙ—]. In addition, saturated, unsaturated, substituted and unsubstituted forms of each of the foregoing are envisioned. Moreover, the end-capping group can also be a silane. The end-capping group can also advantageously comprise a detectable label. When the polymer has an end-capping group comprising a detectable label, the amount or location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, gold particles, quantum dots, and the like. Suitable detectors include photometers, films, spectrometers, and the like. The end-capping group can also advantageously comprise a phospholipid. When the polymer has an end-capping group comprising a phospholipid, unique properties are imparted to the polymer and the resulting conjugate. Exemplary phospholipids include, without limitation, those selected from the class of phospholipids called phosphatidylcholines. Specific phospholipids include, without limitation, those selected from the group consisting of dilauroylphosphatidylcholine, dioleylphosphatidylcholine, dipalmitoylphosphatidylcholine, disteroylphosphatidylcholine, behenoylphosphatidylcholine, arachidoylphosphatidylcholine, and lecithin.

The term “targeting moiety” is used herein to refer to a molecular structure that helps the conjugates of the invention to localize to a targeting area, e.g., help enter, permeate, or penetrate a cell, or bind a receptor. Preferably, the targeting moiety comprises of vitamin, cofactor, antibody, antigen, receptor, DNA, RNA, sialyl Lewis X antigen, hyaluronic acid, sugars, cell specific lectins, steroid or steroid derivative, RGD peptide, cell penetrating or cell targeting moiety, ligand for a cell surface receptor, serum component, or combinatorial molecule directed against various intra- or extracellular receptors. The targeting moiety may also comprise a lipid or a phospholipid. Exemplary phospholipids include, without limitation, phosphatidylcholines, phospatidylserine, phospatidylinositol, phospatidylglycerol, and phospatidylethanolamine. These lipids may be in the form of micelles or liposomes and the like. The targeting moiety may further comprise a detectable label or alternately a detectable label may serve as a targeting moiety. When the conjugate has a targeting group comprising a detectable label, the amount and/or distribution/location of the polymer and/or the moiety (e.g., active agent) to which the polymer is coupled can be determined by using a suitable detector. Such labels include, without limitation, fluorescers, chemiluminescers, moieties used in enzyme labeling, colorimetric (e.g., dyes), metal ions, radioactive moieties, gold particles, quantum dots, and the like.

“Branched,” in reference to the geometry or overall structure of an oligomer, refers to an oligomer having two or more polymer “arms” extending from a branch point.

“Forked,” in reference to the geometry or overall structure of an oligomer, refers to an oligomer having two or more functional groups (typically through one or more atoms) extending from a branch point.

A “branch point” refers to a bifurcation point comprising one or more atoms at which an oligomer branches or forks from a linear structure into one or more additional arms.

The term “reactive” or “activated” refers to a functional group that reacts readily or at a practical rate under conventional conditions of organic synthesis. This is in contrast to those groups that either do not react or require strong catalysts or impractical reaction conditions in order to react (i.e., a “nonreactive” or “inert” group).

“Not readily reactive,” with reference to a functional group present on a molecule in a reaction mixture, indicates that the group remains largely intact under conditions that are effective to produce a desired reaction in the reaction mixture.

A “protecting group” is a moiety that prevents or blocks reaction of a particular chemically reactive functional group in a molecule under certain reaction conditions. The protecting group may vary depending upon the type of chemically reactive group being protected as well as the reaction conditions to be employed and the presence of additional reactive or protecting groups in the molecule. Functional groups which may be protected include, by way of example, carboxylic acid groups, amino groups, hydroxyl groups, thiol groups, carbonyl groups and the like. Representative protecting groups for carboxylic acids include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein.

A functional group in “protected form” refers to a functional group bearing a protecting group. As used herein, the term “functional group” or any synonym thereof encompasses protected forms thereof.

A “physiologically cleavable” or “hydrolyzable” or “degradable” bond is a relatively labile bond that reacts with water (i.e., is hydrolyzed) under physiological conditions. The tendency of a bond to hydrolyze in water may depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, and carbonates.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages include but are not limited to the following: carbon-carbon bonds (e.g., in aliphatic chains), ethers, amides, urethanes, amines, and the like. Generally, a stable linkage is one that exhibits a rate of hydrolysis of less than about 1-2% per day under physiological conditions. Hydrolysis rates of representative chemical bonds can be found in most standard chemistry textbooks.

“Substantially” or “essentially” means nearly totally or completely, for instance, 95% or greater, more preferably 97% or greater, still more preferably 98% or greater, even more preferably 99% or greater, yet still more preferably 99.9% or greater, with 99.99% or greater being most preferred of some given quantity.

“Monodisperse” refers to an oligomer composition wherein substantially all of the oligomers in the composition have a well-defined, single molecular weight and defined number of monomers, as determined by chromatography or mass spectrometry. Monodisperse oligomer compositions are in one sense pure, that is, substantially having a single and definable number (as a whole number) of monomers rather than a large distribution. A monodisperse oligomer composition possesses a MW/Mn value of 1.0005 or less, and more preferably, a MW/Mn value of 1.0000. By extension, a composition comprised of monodisperse conjugates means that substantially all oligomers of all conjugates in the composition have a single and definable number (as a whole number) of monomers rather than a large distribution and would possess a MW/Mn value of 1.0005, and more preferably, a MW/Mn value of 1.0000 if the oligomer were not attached to the therapeutic moiety. A composition comprised of monodisperse conjugates may, however, include one or more nonconjugate substances such as solvents, reagents, excipients, and so forth.

“Bimodal,” in reference to an oligomer composition, refers to an oligomer composition wherein substantially all oligomers in the composition have one of two definable and different numbers (as whole numbers) of monomers rather than a large distribution, and whose distribution of molecular weights, when plotted as a number fraction versus molecular weight, appears as two separate identifiable peaks. Preferably, for a bimodal oligomer composition as described herein, each peak is generally symmetric about its mean, although the size of the two peaks may differ. Ideally, the polydispersity index of each peak in the bimodal distribution, Mw/Mn, is 1.01 or less, more preferably 1.001 or less, and even more preferably 1.0005 or less, and most preferably a MW/Mn value of 1.0000. By extension, a composition comprised of bimodal conjugates means that substantially all oligomers of all conjugates in the composition have one of two definable and different numbers (as whole numbers) of monomers rather than a large distribution and would possess a MW/Mn value of 1.01 or less, more preferably 1.001 or less and even more preferably 1.0005 or less, and most preferably a MW/Mn value of 1.0000 if the oligomer were not attached to the therapeutic moiety. A composition comprised of bimodal conjugates may, however, include one or more nonconjugate substances such as solvents, reagents, excipients, and so forth.

An “antihypertensive” is broadly used herein to refer to an organic, inorganic, or organometallic compound having a molecular weight of less than about 1000 Daltons and having some degree of activity as antihypertensive therapeutic. Antihypertensive activity of a compound may be measured by assays known in the art and also as described herein later.

A “biological membrane” is any membrane made of cells or tissues that serves as a barrier to at least some foreign entities or otherwise undesirable materials. As used herein a “biological membrane” includes those membranes that are associated with physiological protective barriers including, for example: the blood-brain barrier (BBB); the blood-cerebrospinal fluid barrier; the blood-placental barrier; the blood-milk barrier; the blood-testes barrier; and mucosal barriers including the vaginal mucosa, urethral mucosa, anal mucosa, buccal mucosa, sublingual mucosa, and rectal mucosa. Unless the context clearly dictates otherwise, the term “biological membrane” does not include those membranes associated with the middle gastro-intestinal tract (e.g., stomach and small intestines).

A “biological membrane crossing rate,” provides a measure of a compound's ability to cross a biological membrane, such as the blood-brain barrier (“BBB”). A variety of methods may be used to assess transport of a molecule across any given biological membrane. Methods to assess the biological membrane crossing rate associated with any given biological barrier (e.g., the blood-cerebrospinal fluid barrier, the blood-placental barrier, the blood-milk barrier, the intestinal barrier, and so forth), are known, described herein and/or in the relevant literature, and/or may be determined by one of ordinary skill in the art.

A “reduced rate of metabolism” refers to a measurable reduction in the rate of metabolism of a water-soluble oligomer-small molecule drug conjugate as compared to the rate of metabolism of the small molecule drug not attached to the water-soluble oligomer (i.e., the small molecule drug itself) or a reference standard material. In the special case of “reduced first pass rate of metabolism,” the same “reduced rate of metabolism” is required except that the small molecule drug (or reference standard material) and the corresponding conjugate are administered orally. Orally administered drugs are absorbed from the gastro-intestinal tract into the portal circulation and may pass through the liver prior to reaching the systemic circulation. Because the liver is the primary site of drug metabolism or biotransformation, a substantial amount of drug may be metabolized before it ever reaches the systemic circulation. The degree of first pass metabolism, and thus, any reduction thereof, may be measured by a number of different approaches. For instance, animal blood samples may be collected at timed intervals and the plasma or serum analyzed by liquid chromatography/mass spectrometry for metabolite levels. Other techniques for measuring a “reduced rate of metabolism” associated with the first pass metabolism and other metabolic processes are known, described herein and/or in the relevant literature, and/or may be determined by one of ordinary skill in the art. Preferably, a conjugate of the invention may provide a reduced rate of metabolism reduction satisfying at least one of the following values: at least about 30%; at least about 40%; at least about 50%; at least about 60%; at least about 70%; at least about 80%; and at least about 90%. A compound (such as a small molecule drug or conjugate thereof) that is “orally bioavailable” is one that preferably possesses a bioavailability when administered orally of greater than 25%, and preferably greater than 70%, where a compound's bioavailability is the fraction of administered drug that reaches the systemic circulation in unmetabolized form.

“Alkyl” refers to a hydrocarbon chain, ranging from about 1 to 20 atoms in length. Such hydrocarbon chains,are preferably but not necessarily saturated and may be branched or straight chain. Exemplary alkyl groups include methyl, ethyl, propyl, butyl, pentyl, 2-methylbutyl, 2-ethylpropyl, 3-methylpentyl, and the like. As used herein, “alkyl” includes cycloalkyl when three or more carbon atoms are referenced. An “alkenyl” group is an alkyl of 2 to 20 carbon atoms with at least one carbon-carbon double bond.

The terms “substituted alkyl” or “substituted C_{q-r} alkyl” where q and r are integers identifying the range of carbon atoms contained in the alkyl group, denotes the above alkyl groups that are substituted by one, two or three halo (e.g., F, Cl, Br, I), trifluoromethyl, hydroxy, C₁₋₇ alkyl (e.g., methyl, ethyl, n-propyl, isopropyl, butyl, t-butyl, and so forth), C₁₋₇ alkoxy, C₁₋₇ acyloxy, C₃₋₇ heterocyclic, amino, phenoxy, nitro, carboxy, acyl, cyano. The substituted alkyl groups may be substituted once, twice or three times with the same or with different substituents.

“Lower alkyl” refers to an alkyl group containing from 1 to 6 carbon atoms, and may be straight chain or branched, as exemplified by methyl, ethyl, n-butyl, i-butyl, t-butyl. “Lower alkenyl” refers to a lower alkyl group of 2 to 6 carbon atoms having at least one carbon-carbon double bond.

“Non-interfering substituents” are those groups that, when present in a molecule, are typically non-reactive with other functional groups contained within the molecule.

“Alkoxy” refers to an —O—R group, wherein R is alkyl or substituted alkyl, preferably C₁-C₂₀ alkyl (e.g., methoxy, ethoxy, propyloxy, etc.), preferably C₁-C₇.

“Pharmaceutically acceptable excipient” or “pharmaceutically acceptable carrier” refers to component that may be included in the compositions of the invention causes no significant adverse toxicological effects to a patient.

The term “aryl” means an aromatic group having up to 14 carbon atoms. Aryl groups include phenyl, naphthyl, biphenyl, phenanthrenyl, naphthalenyl, and the like. “Substituted phenyl” and “substituted aryl” denote a phenyl group and aryl group, respectively, substituted with one, two, three, four or five (e.g. 1-2,1-3 or 1-4 substituents) chosen from halo (F, Cl, Br, I), hydroxy, cyano, nitro, alkyl (e.g., C₁₋₆ alkyl), alkoxy (e.g., C₁₋₆ alkoxy), benzyloxy, carboxy, aryl, and so forth.

Chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g., CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding multivalent moiety, arylene). All atoms are understood to have their normal number of valences for bond formation (i.e., 1 for H, 4 for carbon, 3 for N, 2 for O, and 2, 4, or 6 for S, depending on the oxidation state of the S).

“Pharmacologically effective amount,” “physiologically effective amount,” and “therapeutically effective amount” are used interchangeably herein to mean the amount of a water-soluble oligomer-small molecule drug conjugate present in a composition that is needed to provide a desired level of active agent and/or conjugate in the bloodstream or in the target tissue. The precise amount may depend upon numerous factors, e.g., the particular active agent, the components and physical characteristics of the composition, intended patient population, patient considerations, and may readily be determined by one skilled in the art, based upon the information provided herein and available in the relevant literature.

A “difunctional” oligomer is an oligomer having two functional groups contained therein, typically at its termini. When the functional groups are the same, the oligomer is said to be homodifunctional. When the functional groups are different, the oligomer is said to be heterodifunctional.

A basic reactant or an acidic reactant described herein include neutral, charged, and any corresponding salt forms thereof.

The term “patient,” refers to a living organism suffering from or prone to a condition that can be prevented or treated by administration of a conjugate as described herein, and includes both humans and animals.

“Optional” or “optionally” means that the subsequently described circumstance may but need not necessarily occur, so that the description includes instances where the circumstance occurs and instances where it does not.

“Nil” refers to the absence of a substituent group. Thus, when a substituent is nil, the substituent may be represented in the structure as a chemical bond or hydrogen in the resulting structure.

As indicated above, the present invention is directed to (among other things) a compound comprising a guanidine class antihypertensive residue covalently attached via a stable or degradable linkage to a water-soluble, non-peptidic oligomer.

The “antihypertensive residue” is a compound having a structure of a guanidine class antihypertensive compound that is altered by the presence of one or more bonds, which bonds serve to attach (either directly or indirectly) one or more water-soluble, non-peptidic oligomers. Exemplary antihypertensives have a structure encompassed by at least one of the structures defined herein as Formula I:

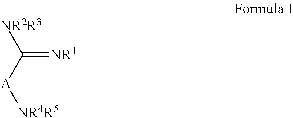

wherein:
A is selected from the group consisting of nil, lower alkylene, —CH₂NH—, and —CH₂CH₂NH—;
R¹ is selected from the group consisting of hydrogen, alkyl, cyano, and aryl;
R² and R³ are each independently selected from the group consisting of hydrogen, hydroxyl, alkyl, substituted alkyl, aryl, substituted aryl, and alkylaryl; and
R⁴ and R⁵ are each independently selected from the group consisting of hydrogen, hydroxyl, alkyl, substituted alkyl, aryl, substituted aryl, heteroaryl and alkylaryl, or R⁴, R⁵, and the nitrogen to which they are attached, together form heteroaryl, or heterocycle.

Examples of specific antihypertensive compounds include those selected from the group consisting of bethanidine, debrisoquin, guanabenz, guanadrel, guanethidine, guanfacine, guanoxabenz, guanoxan, and pinacidil.

It is believed that an advantage of the compounds of the present invention is their ability to retain some degree of antihypertensive activity while also exhibiting a decrease in metabolism. Although not wishing to be bound by theory, it is believed that the antihypertensive residue- and oligomer-containing compounds described herein—in contrast to the oligomer-free “original” antihypertensive structure—are not metabolized as readily because the oligomer serves to reduce the overall affinity of the compound to substrates that may metabolize antihypertensives. In addition (and again, not wishing to be bound by theory), the extra size introduced by the oligomer—in contrast to the oligomer-free “original” antihypertensive structure—reduces the ability of the compound to cross the blood-brain barrier. Even should the linkage between the residue of the antihypertensive and the oligomer be degradable, the compound still offers advantages (such as avoiding first-pass metabolism upon initial absorption).

Use of discrete oligomers (e.g., from a monodisperse or bimodal composition of oligomers, in contrast to relatively impure compositions) to form oligomer-containing compounds may advantageously alter certain properties associated with the corresponding small molecule drug. For instance, a compound of the invention, when administered by any of a number of suitable administration routes, such as parenteral, oral, transdermal, buccal, pulmonary, or nasal, exhibits reduced penetration across the blood-brain barrier. It is preferred that the compounds of the invention exhibit slowed, minimal or effectively no crossing of the blood-brain barrier, while still crossing the gastro-intestinal (GI) walls and into the systemic circulation if oral delivery is intended. Moreover, the compounds of the invention maintain a degree of bioactivity as well as bioavailability in comparison to the bioactivity and bioavailability of the compound free of all oligomers.

With respect to the blood-brain barrier (“BBB”), this barrier restricts the transport of drugs from the blood to the brain. This barrier consists of a continuous layer of unique endothelial cells joined by tight junctions. The cerebral capillaries, which comprise more than 95% of the total surface area of the BBB, represent the principal route for the entry of most solutes and drugs into the central nervous system.

For compounds whose degree of blood-brain barrier crossing ability is not readily known, such ability may be determined using a suitable animal model such as an in situ rat brain perfusion (“RBP”) model as described herein. Briefly, the RBP technique involves cannulation of the carotid artery followed by perfusion with a compound solution under controlled conditions, followed by a wash out phase to remove compound remaining in the vascular space. (Such analyses may be conducted, for example, by contract research organizations such as Absorption Systems, Exton, Pa.). In one example of the RBP model, a cannula is placed in the left carotid artery and the side branches are tied off. A physiologic buffer containing the analyte (typically but not necessarily at a 5 micromolar concentration level) is perfused at a flow rate of about 10 mL/minute in a single pass perfusion experiment. After 30 seconds, the perfusion is stopped and the brain vascular contents are washed out with compound-free buffer for an additional 30 seconds. The brain tissue is then removed and analyzed for compound concentrations via liquid chromatograph with tandem mass spectrometry detection (LC/MS/MS). Alternatively, blood-brain barrier permeability can be estimated based upon a calculation of the compound's molecular polar surface area (“PSA”), which is defined as the sum of surface contributions of polar atoms (usually oxygens, nitrogens and attached hydrogens) in a molecule. The PSA has been shown to correlate with compound transport properties such as blood-brain barrier transport. Methods for determining a compound's PSA can be found, e.g., in, Ertl, P., et al., J. Med. Chem. 2000, 43, 3714-3717; and Kelder, J., et al., Pharm. Res. 1999, 16, 1514-1519.

With respect to the blood-brain barrier, the water-soluble, non-peptidic oligomer-small molecule drug conjugate exhibits a blood-brain barrier crossing rate that is reduced as compared to the crossing rate of the small molecule drug not attached to the water-soluble, non-peptidic oligomer. Exemplary reductions in blood-brain barrier crossing rates for the compounds described herein include reductions of: at least about 5%; at least about 10%; at least about 25%; at least about 30%; at least about 40%; at least about 50%; at least about 60%; at least about 70%; at least about 80%; or at least about 90%, when compared to the blood-brain barrier crossing rate of the small molecule drug not attached to the water-soluble oligomer. A preferred reduction in the blood-brain barrier crossing rate for a conjugate of the invention is at least about 20%.

As indicated above, the compounds of the invention include a guanidine class antihypertensive residue. Assays for determining whether a given compound (regardless of whether the compound includes a water-soluble, non-peptidic oligomer or not) can act as a guanidine class antihypertensive are described infra.

The variables, notations, and symbols used in the following paragraphs with respect to formula may not relate to other paragraphs. Therefore, definitions of the notations and symbols in each paragraph are normally limited to it and should not be used to construe other paragraphs, unless indicated otherwise.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula: N-benzyl-N′,N″-dimethylguanidine, N-2,4-dichlorobenzylguanidine, N,N′-dimethyl-N″-3-methylbenzylguanidine, N-2-chlorobenzyl-N′,N″-dimethylguanidine, N,N′-dimethyl-N″-2-methylbenzylguanidine, N-3-chlorobenzyl-N′,N″-dimethylguanidine, N-2-chlorobenzyl-N′-methylguanidine, and N-ethyl-N′-methyl-N″-2-methylbenzylguanidine.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

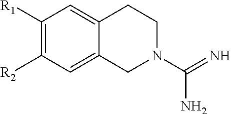

wherein:
R₁ and R₂ are individually selected from the group consisting of hydrogen, lower alkoxy, and taken together, lower alkylenedioxy.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

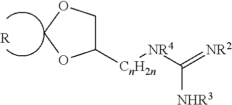

wherein:
n is an integer from 1-8; R², R³, and R⁴ each are members of the group consisting of hydrogen and lower alkyl; and R is an alkylene group containing 4 to 9 carbon atoms in the chain, with the proviso that when the alkylene group contains 4-5 carbon atoms, the chain can have fused thereto from 1 to 2 benzo rings, or the corresponding alkylene group substituted with up to 4 members of the group consisting of halo, lower alkyl, trifluoromethyl, trichloromethyl, lower alkoxy, benzyloxy, phenoxy, phenyl, hydroxy, carboxy, carbo-lower-alkoxy, nitro, sulfato and acetamido, said R group having a molecular weight less than 300.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

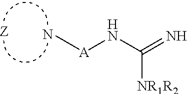

in which

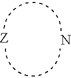

represents N,N-alkylene-imino, the alkylene portion of which contains from four to ten carbon atoms, A stands for lower alkylene containing from one to five carbon atoms, R₁ represents a member of the group consisting of hydrogen and lower alkyl, R₂ represents a member of the group consisting of hydrogen, lower alkyl, lower alkanoyl, benzoyl and methoxy-substituted benzoyl, and R₃ stands for a member of the group consisting of hydrogen and lower alkyl.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

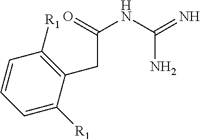

in which R₁ is chloro or methyl.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

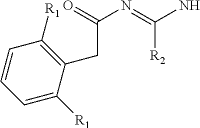

in which R₁ is chloro or methyl and R₂ is lower alkoxy, lower alkylthio, or p-nitrobenzylthio.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

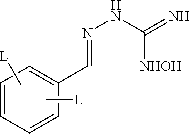

where each L, independently, represents hydrogen or halo having an atomic weight of about 19-36, provided at least one L is other than hydrogen.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

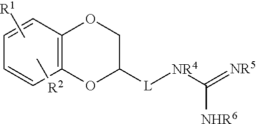

wherein each R¹ and R² is selected from the group consisting of hydrogen, chloro, bromo, lower alkyl and lower alkoxy; L is selected from the group consisting of lower alkylene, —CH₂CH₂NH— and —CH₂NH—; R⁴ is selected from the group consisting of hydrogen and lower alkyl and R⁵ and R⁶, when taken together with the nitrogen atoms to which they are attached and the carbon atom which is geminal to said nitrogen atoms, are imidazolinyl.

In one or more embodiments of the invention, a compound is provided, the compound comprising a guanidine class antihypertensive residue covalently attached via a stable or degradable linkage to a water-soluble, non-peptidic oligomer, wherein the antihypertensive has a structure encompassed by the following formula:

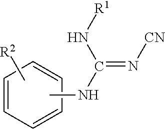

in which the R¹-substituted cyano-guanidyl radical is placed in the 2-, 3-, or 4-position of the pyridine ring, and in which R¹ stands for aliphatic hydrocarbon having from 1 to 8 carbon atoms, cycloalkyl having from 3 to 8 carbon atoms, phenyl, benzyl, or phenethyl, and R² stands for hydrogen, halo, hydroxyl, lower alkyl or lower alkoxy radicals.

In one or more embodiments of the invention, a compound is provided, the compound comprising a guanidine class antihypertensive residue covalently attached via a stable or degradable linkage to a water-soluble, non-peptidic oligomer, wherein the guanidine class antihypertensive is selected from the group consisting of bethanidine, debrisoquin, guanabenz, guanadrel, guanathidine, guanafacine, guanoxabenz, guanoxan, and pinacidil.

In some instances, antihypertensives can be obtained from commercial sources. In addition, antihypertensives can be obtained through chemical synthesis. Examples of antihypertensives as well as synthetic approaches for preparing antihypertensives are described in the literature and in, for example, U.S. Pat. No. 3,168,562, U.S. Pat. No. 3,157,573, U.S. Pat. No. 3,547,951, U.S. Pat. No. 2,928,829, U.S. Pat. No. 3,632,645, U.S. Pat. No. 3,591,636, U.S. Pat. No. 3,247,221, and U.S. Pat. No. 4,057,636.

Each of these (and other) antihypertensives can be covalently attached (either directly or through one or more atoms) to a water-soluble, non-peptidic oligomer.

Small molecule drugs useful in the invention generally have a molecular weight of less than 1000 Da. Exemplary molecular weights of small molecule drugs include molecular weights of: less than about 950; less than about 900; less than about 850; less than about 800; less than about 750; less than about 700; less than about 650; less than about 600; less than about 550; less than about 500; less than about 450; less than about 400; less than about 350; and less than about 300.

The small molecule drug used in the invention, if chiral, may be obtained from a racemic mixture, or an optically active form, for example, a single optically active enantiomer, or any combination or ratio of enantiomers (i.e., scalemic mixture). In addition, the small molecule drug may possess one or more geometric isomers. With respect to geometric isomers, a composition can comprise a single geometric isomer or a mixture of two or more geometric isomers. A small molecule drug for use in the present invention can be in its customary active form, or may possess some degree of modification. For example, a small molecule drug may have a targeting agent, tag, or transporter attached thereto, prior to or after covalent attachment of an oligomer. Alternatively, the small molecule drug may possess a lipophilic moiety attached thereto, such as a phospholipid (e.g., distearoylphosphatidylethanolamine or “DSPE,” dipalmitoylphosphatidylethanolamine or “DPPE,” and so forth) or a small fatty acid. In some instances, however, it is preferred that the small molecule drug moiety does not include attachment to a lipophilic moiety.

The parent drug molecule for coupling to a water-soluble, non-peptidic oligomer possesses a free hydroxyl, carboxyl, thio, amino group, or the like (i.e., “handle”) suitable for covalent attachment to the oligomer. In addition, the antihypertensive may be modified by introduction of a reactive group, preferably by conversion of one of its existing functional groups to a functional group suitable for formation of a stable covalent linkage between the oligomer and the drug. Both approaches are illustrated in the Experimental section.

Accordingly, each oligomer is composed of up to three different monomer types selected from the group consisting of: alkylene oxide, such as ethylene oxide or propylene oxide; olefinic alcohol, such as vinyl alcohol, 1-propenol or 2-propenol; vinyl pyrrolidone; hydroxyalkyl methacrylamide or hydroxyalkyl methacrylate, where alkyl is preferably methyl; α-hydroxy acid, such as lactic acid or glycolic acid; phosphazene, oxazoline, amino acids, carbohydrates such as monosaccharides, alditol such as mannitol; and N-acryloylmorpholine. Preferred monomer types include alkylene oxide, olefinic alcohol, hydroxyalkyl methacrylamide or methacrylate, N-acryloylmorpholine, and α-hydroxy acid. Preferably, each oligomer is, independently, a co-oligomer of two monomer types selected from this group, or, more preferably, is a homo-oligomer of one monomer type selected from this group.

The two monomer types in a co-oligomer may be of the same monomer type, for example, two alkylene oxides, such as ethylene oxide and propylene oxide. Preferably, the oligomer is a homo-oligomer of ethylene oxide. Usually, although not necessarily, the terminus (or termini) of the oligomer that is not covalently attached to a small molecule is capped to render it unreactive. Alternatively, the terminus may include a reactive group. When the terminus is a reactive group, the reactive group is either selected such that it is unreactive under the conditions of formation of the final oligomer or during covalent attachment of the oligomer to a small molecule drug, or it is protected as necessary. One common end-functional group is hydroxyl or —OH, particularly for oligoethylene oxides.

The water-soluble, non-peptidic oligomer (e.g., “POLY” in various structures provided herein) can have any of a number of different geometries. For example, the water-soluble, non-peptidic oligomer may be linear, branched, or forked. Most typically, the water-soluble, non-peptidic oligomer is linear or is branched, for example, having one branch point. Although much of the discussion herein is focused upon poly(ethylene oxide) as an illustrative oligomer, the discussion and structures presented herein can be readily extended to encompass any water-soluble, non-peptidic oligomers described above.

The molecular weight of the water-soluble, non-peptidic oligomer, excluding the linker portion, is generally relatively low. Exemplary values of the molecular weight of the water-soluble polymer include: below about 1500; below about 1450; below about 1400; below about 1350; below about 1300; below about 1250; below about 1200; below about 1150; below about 1100; below about 1050; below about 1000; below about 950; below about 900; below about 850; below about 800; below about 750; below about 700; below about 650; below about 600; below about 550; below about 500; below about 450; below about 400; below about 350; below about 300; below about 250; below about 200; and below about 100 Daltons.

Exemplary ranges of molecular weights of the water-soluble, non-peptidic oligomer (excluding the linker) include: from about 100 to about 1400 Daltons; from about 100 to about 1200 Daltons; from about 100 to about 800 Daltons; from about 100 to about 500 Daltons; from about 100 to about 400 Daltons; from about 200 to about 500 Daltons; from about 200 to about 400 Daltons; from about 75 to 1000 Daltons; and from about 75 to about 750 Daltons.

Preferably, the number of monomers in the water-soluble, non-peptidic oligomer falls within one or more of the following ranges: between about 1 and about 30 (inclusive); between about 1 and about 25; between about 1 and about 20; between about 1 and about 15; between about 1 and about 12; between about 1 and about 10. In certain instances, the number of monomers in series in the oligomer (and the corresponding conjugate) is one of 1, 2, 3, 4, 5, 6, 7, or 8. In additional embodiments, the oligomer (and the corresponding conjugate) contains 9, 10, 11, 12, 13, 14, 15, 16, 17, 18, 19, or 20 monomers. In yet further embodiments, the oligomer (and the corresponding conjugate) possesses 21, 22, 23, 24, 25, 26, 27, 28, 29 or 30 monomers in series. Thus, for example, when the water-soluble, non-peptidic polymer includes CH₃—(OCH₂CH₂)ₙ—, “n” is an integer that can be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, and can fall within one or more of the following ranges: between about 1 and about 25; between about 1 and about 20; between about 1 and about 15; between about 1 and about 12; between about 1 and about 10.

When the water-soluble, non-peptidic oligomer has 1, 2, 3, 4, 5, 6, 7, 8, 9, or 10 monomers, these values correspond to a methoxy end-capped oligo(ethylene oxide) having a molecular weights of about 75, 119, 163, 207, 251, 295, 339, 383, 427, and 471 Daltons, respectively. When the oligomer has 11, 12, 13, 14, or 15 monomers, these values correspond to methoxy end-capped oligo(ethylene oxide) having molecular weights corresponding to about 515, 559, 603, 647, and 691 Daltons, respectively.

When the water-soluble, non-peptidic oligomer is attached to the antihypertensive (in contrast to the step-wise addition of one or more monomers to effectively “grow” the oligomer onto the antihypertensive), it is preferred that the composition containing an activated form of the water-soluble, non-peptidic oligomer be monodisperse. In those instances, however, where a bimodal composition is employed, the composition will possess a bimodal distribution centering around any two of the above numbers of monomers. For instance, a bimodal oligomer may have any one of the following exemplary combinations of monomer subunits: 1-2, 1-3, 1-4, 1-5, 1-6, 1-7, 1-8, 1-9, 1-10, and so forth; 2-3, 2-4, 2-5, 2-6, 2-7, 2-8, 2-9, 2-10, and so forth; 3-4, 3-5, 3-6, 3-7, 3-8, 3-9, 3-10, and so forth; 4-5, 4-6, 4-7, 4-8, 4-9, 4-10, and so forth; 5-6, 5-7, 5-8, 5-9, 5-10, and so forth; 6-7, 6-8, 6-9, 6-10, and so forth; 7-8, 7-9, 7-10, and so forth; and 8-9, 8-10, and so forth.

In some instances, the composition containing an activated form of the water-soluble, non-peptidic oligomer will be trimodal or even tetramodal, possessing a range of monomers units as previously described. Oligomer compositions possessing a well-defined mixture of oligomers (i.e., being bimodal, trimodal, tetramodal, and so forth) can be prepared by mixing purified monodisperse oligomers to obtain a desired profile of oligomers (a mixture of two oligomers differing only in the number of monomers is bimodal; a mixture of three oligomers differing only in the number of monomers is trimodal; a mixture of four oligomers differing only in the number of monomers is tetramodal), or alternatively, can be obtained from column chromatography of a polydisperse oligomer by recovering the “center cut”, to obtain a mixture of oligomers in a desired and defined molecular weight range.

It is preferred that the water-soluble, non-peptidic oligomer is obtained from a composition that is preferably unimolecular or monodisperse. That is, the oligomers in the composition possess the same discrete molecular weight value rather than a distribution of molecular weights. Some monodisperse oligomers can be purchased from commercial sources such as those available from Sigma-Aldrich, or alternatively, can be prepared directly from commercially available starting materials such as Sigma-Aldrich. Water-soluble, non-peptidic oligomers can be prepared as described in Chen Y., Baker, G. L., J. Org. Chem., 6870-6873 (1999), WO 02/098949, and U.S. Patent Application Publication 2005/0136031.

When present, the spacer moiety (through which the water-soluble, non-peptidic polymer is attached to the antihypertensive) may be a single bond, a single atom, such as an oxygen atom or a sulfur atom, two atoms, or a number of atoms. A spacer moiety is typically but is not necessarily linear in nature. The spacer moiety, “X,” is hydrolytically stable, and is preferably also enzymatically stable. Preferably, the spacer moiety “X” is one having a chain length of less than about 12 atoms, and preferably less than about 10 atoms, and even more preferably less than about 8 atoms and even more preferably less than about 5 atoms, whereby length is meant the number of atoms in a single chain, not counting substituents. For instance, a urea linkage such as this, R_{oligomer}—NH—(C═O)—NH—R′_{drug}, is considered to have a chain length of 3 atoms (—NH—C(O)—NH—). In selected embodiments, the linkage does not comprise further spacer groups.

In some instances, the spacer moiety “X” comprises an ether, amide, urethane, amine, thioether, urea, or a carbon-carbon bond. Functional groups such as those discussed below, and illustrated in the examples, are typically used for forming the linkages. The spacer moiety may less preferably also comprise (or be adjacent to or flanked by) other atoms, as described further below.

More specifically, in selected embodiments, a spacer moiety of the invention, X, may be any of the following: “—” (i.e., a covalent bond, that may be stable or degradable, between the antihypertensive residue and the water-soluble, non-peptidic oligomer),), —O—, —NH—, —S—, —C(O)—, —C(O)O—, —OC(O)—, —CH₂—C(O)O—, —CH₂—OC(O)—, —C(O)O—CH₂—, —OC(O)—CH₂—, C(O)—NH, NH—C(O)—NH, O—C(O)—NH, —C(S)—, —CH₂—, —CH₂—CH₂—, —CH₂—CH₂—CH₂—, —CH₂—CH₂—CH₂—CH₂—, —O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂, —CH₂—CH₂—NH—C(O)—CH₂—CH₂, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂NH—C(O)—, —CH₂—CH₂—CH₂—C(O)—NH—CH₂—CH₂—NH—C(O)—CH₂—, bivalent cycloalkyl group, —N(R⁶)—, R⁶ is H or an organic radical selected from the group consisting of alkyl, substituted alkyl, alkenyl, substituted alkenyl, alkynyl, substituted alkynyl, aryl and substituted aryl. Additional spacer moieties include, acylamino, acyl, aryloxy, alkylene bridge containing between 1 and 5 inclusive carbon atoms, alkylamino, dialkylamino having about 2 to 4 inclusive carbon atoms, piperidino, pyrrolidino, N-(lower alkyl)-2-piperidyl, morpholino, 1-piperizinyl, 4-(lower alkyl)-1-piperizinyl, 4-(hydroxyl-lower alkyl)-1-piperizinyl, 4-(methoxy-lower alkyl)-1-piperizinyl, and guanidine.

For purposes of the present invention, however, a group of atoms is not considered a linkage when it is immediately adjacent to an oligomer segment, and the group of atoms is the same as a monomer of the oligomer such that the group would represent a mere extension of the oligomer chain.

The linkage “X” between the water-soluble, non-peptidic oligomer and the small molecule is typically formed by reaction of a functional group on a terminus of the oligomer (or nascent oligomer when it is desired to “grow” the oligomer onto the antihypertensive) with a corresponding functional group within the antihypertensive. Illustrative reactions are described briefly below. For example, an amino group on an oligomer may be reacted with a carboxylic acid or an activated carboxylic acid derivative on the small molecule, or vice versa, to produce an amide linkage. Alternatively, reaction of an amine on an oligomer with an activated carbonate (e.g. succinimidyl or benzotriazyl carbonate) on the drug, or vice versa, forms a carbamate linkage. Reaction of an amine on an oligomer with an isocyanate (R—N═C═O) on a drug, or vice versa, forms a urea linkage (R—NH—(C═O)—NH—R′). Further, reaction of an alcohol (alkoxide) group on an oligomer with an alkyl halide, or halide group within a drug, or vice versa, forms an ether linkage. In yet another coupling approach, a small molecule having an aldehyde function is coupled to an oligomer amino group by reductive amination, resulting in formation of a secondary amine linkage between the oligomer and the small molecule.

A particularly preferred water-soluble, non-peptidic oligomer is an oligomer bearing an aldehyde functional group. In this regard, the oligomer will have the following structure: CH₃O—(CH₂—CH₂—O)ₙ—(CH₂)ₚ—C(O)H, wherein (n) is one of 1, 2, 3, 4, 5, 6, 7, 8, 9 and 10 and (p) is one of 1, 2, 3, 4, 5, 6 and 7. Preferred (n) values include 3, 5 and 7 and preferred (p) values 2, 3 and 4.

The termini of the water-soluble, non-peptidic oligomer not bearing a functional group is capped to render it unreactive. When the oligomer includes a further functional group at a terminus other than that intended for formation of a conjugate, that group is either selected such that it is unreactive under the conditions of formation of the linkage “X,” or it is protected during the formation of the linkage “X.”

As stated above, the water-soluble, non-peptidic oligomer includes at least one functional group prior to conjugation. The functional group typically comprises an electrophilic or nucleophilic group for covalent attachment to a small molecule, depending upon the reactive group contained within or introduced into the small molecule. Examples of nucleophilic groups that may be present in either the oligomer or the small molecule include hydroxyl, amine, hydrazine (—NHNH₂), hydrazide (—C(O)NHNH₂), and thiol. Preferred nucleophiles include amine, hydrazine, hydrazide, and thiol, particularly amine. Most small molecule drugs for covalent attachment to an oligomer will possess a free hydroxyl, amino, thio, aldehyde, ketone, or carboxyl group.

Examples of electrophilic functional groups that may be present in either the oligomer or the small molecule include carboxylic acid, carboxylic ester, particularly imide esters, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, and halosilane. More specific examples of these groups include succinimidyl ester or carbonate, imidazoyl ester or carbonate, benzotriazole ester or carbonate, vinyl sulfone, chloroethylsulfone, vinylpyridine, pyridyl disulfide, iodoacetamide, glyoxal, dione, mesylate, tosylate, and tresylate (2,2,2-trifluoroethanesulfonate).

Also included are sulfur analogs of several of these groups, such as thione, thione hydrate, thioketal, is 2-thiazolidine thione, etc., as well as hydrates or protected derivatives of any of the above moieties (e.g. aldehyde hydrate, hemiacetal, acetal, ketone hydrate, hemiketal, ketal, thioketal, thioacetal).

An “activated derivative” of a carboxylic acid refers to a carboxylic acid derivative that reacts readily with nucleophiles, generally much more readily than the underivatized carboxylic acid. Activated carboxylic acids include, for example, acid halides (such as acid chlorides), anhydrides, carbonates, and esters. Such esters include imide esters, of the general form —(CO)O—N[(CO)—]₂; for example, N-hydroxysuccinimidyl (NHS) esters or N-hydroxyphthalimidyl esters. Also preferred are imidazolyl esters and benzotriazole esters. Particularly preferred are activated propionic acid or butanoic acid esters, as described in co-owned U.S. Pat. No. 5,672,662. These include groups of the form —(CH₂)₂₋₃C(═O)O-Q, where Q is preferably selected from N-succinimide, N-sulfosuccinimide, N-phthalimide, N-glutarimide, N-tetrahydrophthalimide, N-norbornene-2,3-dicarboximide, benzotriazole, 7-azabenzotriazole, and imidazole.

Other preferred electrophilic groups include succinimidyl carbonate, maleimide, benzotriazole carbonate, glycidyl ether, imidazoyl carbonate, p-nitrophenyl carbonate, acrylate, tresylate, aldehyde, and orthopyridyl disulfide.

These electrophilic groups are subject to reaction with nucleophiles, e.g., hydroxy, thio, or amino groups, to produce various bond types. Preferred for the present invention are reactions which favor formation of a hydrolytically stable linkage. For example, carboxylic acids and activated derivatives thereof, which include orthoesters, succinimidyl esters, imidazolyl esters, and benzotriazole esters, react with the above types of nucleophiles to form esters, thioesters, and amides, respectively, of which amides are the most hydrolytically stable. Carbonates, including succinimidyl, imidazolyl, and benzotriazole carbonates, react with amino groups to form carbamates. Isocyanates (R—N═C═O) react with hydroxyl or amino groups to form, respectively, carbamate (RNH—C(O)—OR′) or urea (RNH—C(O)—NHR′) linkages. Aldehydes, ketones, glyoxals, diones and their hydrates or alcohol adducts (i.e., aldehyde hydrate, hemiacetal, acetal, ketone hydrate, hemiketal, and ketal) are preferably reacted with amines, followed by reduction of the resulting imine, if desired, to provide an amine linkage (reductive amination).

Several of the electrophilic functional groups include electrophilic double bonds to which nucleophilic groups, such as thiols, can be added, to form, for example, thioether bonds. These groups include maleimides, vinyl sulfones, vinyl pyridine, acrylates, methacrylates, and acrylamides. Other groups comprise leaving groups that can be displaced by a nucleophile; these include chloroethyl sulfone, pyridyl disulfides (which include a cleavable S—S bond), iodoacetamide, mesylate, tosylate, thiosulfonate, and tresylate. Epoxides react by ring opening by a nucleophile, to form, for example, an ether or amine bond. Reactions involving complementary reactive groups such as those noted above on the oligomer and the small molecule are utilized to prepare the conjugates of the invention.

In some instances the antihypertensive may not have a functional group suited for conjugation. In this instance, it is possible to modify (or “functionalize”) the “original” antihypertensive so that it does have a functional group suited for conjugation. For example, if the antihypertensive has an amide group, but an amine group is desired, it is possible to modify the amide group to an amine group by way of a Hofmann rearrangement, Curtius rearrangement (once the amide is converted to an azide) or Lossen rearrangement (once amide is concerted to hydroxamide followed by treatment with tolyene-2-sulfonyl chloride/base).

It is possible to prepare a conjugate of small molecule antihypertensive bearing a carboxyl group wherein the carboxyl group-bearing small molecule antihypertensive is coupled to an amino-terminated oligomeric ethylene glycol, to provide a conjugate having an amide group covalently linking the small molecule antihypertensive to the oligomer. This can be performed, for example, by combining the carboxyl group-bearing small molecule antihypertensive with the amino-terminated oligomeric ethylene glycol in the presence of a coupling reagent, (such as dicyclohexylcarbodiimide or “DCC”) in an anhydrous organic solvent.

Further, it is possible to prepare a conjugate of a small molecule antihypertensive bearing a hydroxyl group wherein the hydroxyl group-bearing small molecule antihypertensive is coupled to an oligomeric ethylene glycol halide to result in an ether (—O—) linked small molecule conjugate. This can be performed, for example, by using sodium hydride to deprotonate the hydroxyl group followed by reaction with a halide-terminated oligomeric ethylene glycol.

Further, it is possible to prepare a conjugate of a small molecule antihypertensive moiety bearing a hydroxyl group (such as, for example, the antihypertensive moieties having structures encompassed within Formula I) wherein the hydroxyl group-bearing small molecule antihypertensive moiety is coupled to an oligomeric ethylene glycol bearing an haloformate group [e.g., CH₃(OCH₂CH₂)ₙOC(O)-halo, where halo is chloro, bromo, iodo] to result in a carbonate [—O—C(O)—O—] linked small molecule conjugate. This can be performed, for example, by combining a guanidine class antihypertensive moiety and an oligomeric ethylene glycol bearing a haloformate group in the presence of a nucleophilic catalyst (such as 4-dimethylaminopyridine or “DMAP”) to thereby result in the corresponding carbonate-linked conjugate.

In another example, it is possible to prepare a conjugate of a small molecule antihypertensive bearing a ketone group by first reducing the ketone group to form the corresponding hydroxyl group. Thereafter, the small molecule antihypertensive now bearing a hydroxyl group can be coupled as described herein.

In still another instance, it is possible to prepare a conjugate of a small molecule antihypertensive bearing an amine group. In one approach, the amine group-bearing small molecule antihypertensive and an aldehyde-bearing oligomer are dissolved in a suitable buffer after which a suitable reducing agent (e.g., NaCNBH₃) is added. Following reduction, the result is an amine linkage formed between the amine group of the amine group-containing small molecule antihypertensive and the carbonyl carbon of the aldehyde-bearing oligomer.

In another approach for preparing a conjugate of a small molecule antihypertensive bearing an amine group, a carboxylic acid-bearing oligomer and the amine group-bearing small molecule antihypertensive are combined, typically in the presence of a coupling reagent (e.g., DCC). The result is an amide linkage formed between the amine group of the amine group-containing small molecule antihypertensive and the carbonyl of the carboxylic acid-bearing oligomer.

Exemplary compounds of the invention include those having the following structure:

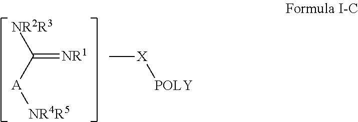

wherein:
A is selected from the group consisting of nil, lower alkylene, —CH2NH—, and —CH2CH2NH—;
R¹ is selected from the group consisting of hydrogen, alkyl, cyano, and aryl;
R² and R³ are each independently selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

Exemplary compounds also include compounds with the following structure:

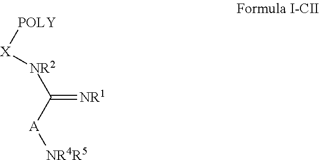

wherein:
A is selected from the group consisting of nil, lower alkylene, —CH₂NH—, and —CH₂CH₂NH—;
R¹ is selected from the group consisting of hydrogen, alkyl, cyano, and aryl;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

Exemplary compounds also include compounds with the following structure:

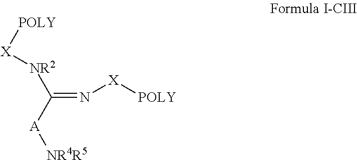

wherein:
A is selected from the group consisting of nil, lower alkylene, —CH₂NH—, and —CH₂CH₂NH—;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl, or heterocycle;
X is a spacer moiety; and
POLY is a water-soluble, non-peptidic oligomer.

While it is believed that the full scope of the conjugates disclosed herein behave as described, an optimally sized oligomer can be identified as follows.

First, an oligomer obtained from a monodisperse or bimodal water soluble oligomer is conjugated to the small molecule drug. Preferably, the drug is orally bioavailable, and on its own, exhibits a non-negligible blood-brain barrier crossing rate. Next, the ability of the conjugate to cross the blood-brain barrier is determined using an appropriate model and compared to that of the unmodified parent drug. If the results are favorable, that is to say, if, for example, the rate of crossing is significantly reduced, then the bioactivity of conjugate is further evaluated. Preferably, the compounds according to the invention maintain a significant degree of bioactivity relative to the parent drug, i.e., greater than about 30% of the bioactivity of the parent drug, or even more preferably, greater than about 50% of the bioactivity of the parent drug.

The above steps are repeated one or more times using oligomers of the same monomer type but having a different number of subunits and the results compared.

For each conjugate whose ability to cross the blood-brain barrier is reduced in comparison to the non-conjugated small molecule drug, its oral bioavailability is then assessed. Based upon these results, that is to say, based upon the comparison of conjugates of oligomers of varying size to a given small molecule at a given position or location within the small molecule, it is possible to determine the size of the oligomer most effective in providing a conjugate having an optimal balance between reduction in biological membrane crossing, oral bioavailability, and bioactivity. The small size of the oligomers makes such screenings feasible and allows one to effectively tailor the properties of the resulting conjugate. By making small, incremental changes in oligomer size and utilizing an experimental design approach, one can effectively identify a conjugate having a favorable balance of reduction in biological membrane crossing rate, bioactivity, and oral bioavailability. In some instances, attachment of an oligomer as described herein is effective to actually increase oral bioavailability of the drug.

For example, one of ordinary skill in the art, using routine experimentation, can determine a best suited molecular size and linkage for improving oral bioavailability by first preparing a series of oligomers with different weights and functional groups and then obtaining the necessary clearance profiles by administering the conjugates to a patient and taking periodic blood and/or urine sampling. Once a series of clearance profiles have been obtained for each tested conjugate, a suitable conjugate can be identified.

Animal models (rodents and dogs) can also be used to study oral drug transport. In addition, non-in vivo methods include rodent everted gut excised tissue and Caco-2 cell monolayer tissue-culture models. These models are useful in predicting oral drug bioavailability.

To determine whether the antihypertensive or the conjugate of a guanidine class antihypertensive and a water-soluble non-peptidic polymer has activity as a guanidine class antihypertensive therapeutic, it is possible to test such a compound. The antihypertensive compounds may be tested using carotid occlusion, nictitating membrane test, and norepinephrine depletion test.

In the carotid occlusion test, a dog (or other suitable animal), is anesthetized with 20 mg/kg of thiopental and maintained with 60 mg/kg of chloralose by intravenous injection. The femoral arteries and veins are catheterized and the common carotid arteries are isolated for bilateral clamping. After determining the control arterial pressor response to 30 sec of carotid occlusion, the test compound is administered and the response is measured again. The dose which produces approximately 40% inhibition of the pressor response is used for potency comparison with other drugs.

In the norepinephrine depletion test, albino guinea pigs (or other suitable animals), weighing 200 to 300 g. are treated with the drug in three test groups, i.e., control, 5 mg/kg I.P., and 10 mg/kg I.P., and then sacrificed after 15 to 18 hrs. Quickly the hearts removed, flushed with saline, placed in a vial and frozen in a dry ice-acetone bath. The hearts are analyzed for norepinephrine by the method of J. R. Crout, J. Pharmacol. 132, 269 (1961), and the level of depletion below control levels are compared with other drugs.

In the nictitating membrane test, a cat (or other suitable animal), is anesthetized with Dialurethane (0.9 ml/kg), with the femoral artery and vein catheterized. The nictitating membrane is drawn out and attached to a thread fastened to a force displacement transducer. The pre-ganglionic nerve to the superior cervical ganglion is isolated for stimulation by supra maximal electrical shock. After determining the control response to pre-ganglionic stimulation, the drug is administered by intravenous injection and the dose which produces at least 10% reduction in response is used for potency comparison with other drugs.

In vitro binding studies to receptors using various cell lines have become routine in pharmaceutical industry.

The present invention also includes pharmaceutical preparations comprising a conjugate as provided herein in combination with a pharmaceutical excipient. Generally, the conjugate itself will be in a solid form (e.g., a precipitate), which can be combined with a suitable pharmaceutical excipient that can be in either solid or liquid form.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, maltitol, lactitol, xylitol, sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines, fatty acids and fatty esters; steroids, such as cholesterol; and chelating agents, such as EDTA, zinc and other such suitable cations.

Pharmaceutically acceptable acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the conjugate in the composition will vary depending on a number of factors, but will optimally be a therapeutically effective dose when the composition is stored in a unit dose container. A therapeutically effective dose can be determined experimentally by repeated administration of increasing amounts of the conjugate in order to determine which amount produces a clinically desired endpoint.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, excipients will be present in the composition in an amount of about 1% to about 99% by weight, preferably from about 5%-98% by weight, more preferably from about 15-95% by weight of the excipient, with concentrations less than 30% by weight most preferred.

These foregoing pharmaceutical excipients along with other excipients and general teachings regarding pharmaceutical compositions are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The pharmaceutical compositions can take any number of forms and the invention is not limited in this regard. Exemplary preparations are most preferably in a form suitable for oral administration such as a tablet, caplet, capsule, gel cap, troche, dispersion, suspension, solution, elixir, syrup, lozenge, transdermal patch, spray, suppository, and powder.

Oral dosage forms are preferred for those conjugates that are orally active, and include tablets, caplets, capsules, gel caps, suspensions, solutions, elixirs, and syrups, and can also comprise a plurality of granules, beads, powders or pellets that are optionally encapsulated. Such dosage forms are prepared using conventional methods known to those in the field of pharmaceutical formulation and described in the pertinent texts.

Tablets and caplets, for example, can be manufactured using standard tablet processing procedures and equipment. Direct compression and granulation techniques are preferred when preparing tablets or caplets containing the conjugates described herein. In addition to the conjugate, the tablets and caplets will generally contain inactive, pharmaceutically acceptable carrier materials such as binders, lubricants, disintegrants, fillers, stabilizers, surfactants, coloring agents, flow agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethylcellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage forms, in which case the conjugate-containing composition can be encapsulated in the form of a liquid or gel (e.g., in the case of a gel cap) or solid (including particulates such as granules, beads, powders or pellets). Suitable capsules include hard and soft capsules, and are generally made of gelatin, starch, or a cellulosic material. Two-piece hard gelatin capsules are preferably sealed, such as with gelatin bands or the like.

Included are parenteral formulations in the substantially dry form (typically as a lyophilizate or precipitate, which can be in the form of a powder or cake), as well as formulations prepared for injection, which are typically liquid and requires the step of reconstituting the dry form of parenteral formulation. Examples of suitable diluents for reconstituting solid compositions prior to injection include bacteriostatic water for injection, dextrose 5% in water, phosphate-buffered saline, Ringer's solution, saline, sterile water, deionized water, and combinations thereof.

In some cases, compositions intended for parenteral administration can take the form of nonaqueous solutions, suspensions, or emulsions, each typically being sterile. Examples of nonaqueous solvents or vehicles are propylene glycol, polyethylene glycol, vegetable oils, such as olive oil and corn oil, gelatin, and injectable organic esters such as ethyl oleate.

The parenteral formulations described herein can also contain adjuvants such as preserving, wetting, emulsifying, and dispersing agents. The formulations are rendered sterile by incorporation of a sterilizing agent, filtration through a bacteria-retaining filter, irradiation, or heat.

The conjugate can also be administered through the skin using conventional transdermal patch or other transdermal delivery system, wherein the conjugate is contained within a laminated structure that serves as a drug delivery device to be affixed to the skin. In such a structure, the conjugate is contained in a layer, or “reservoir,” underlying an upper backing layer. The laminated structure can contain a single reservoir, or it can contain multiple reservoirs.

The conjugate can also be formulated into a suppository for rectal administration. With respect to suppositories, the conjugate is mixed with a suppository base material which is (e.g., an excipient that remains solid at room temperature but softens, melts or dissolves at body temperature) such as coca butter (theobroma oil), polyethylene glycols, glycerinated gelatin, fatty acids, and combinations thereof. Suppositories can be prepared by, for example, performing the following steps (not necessarily in the order presented): melting the suppository base material to form a melt; incorporating the conjugate (either before or after melting of the suppository base material); pouring the melt into a mold; cooling the melt (e.g., placing the melt-containing mold in a room temperature environment) to thereby form suppositories; and removing the suppositories from the mold.

The invention also provides a method for administering a conjugate as provided herein to a patient suffering from a condition that is responsive to treatment with the conjugate. The method comprises administering, generally orally, a therapeutically effective amount of the conjugate (preferably provided as part of a pharmaceutical preparation). Other modes of administration are also contemplated, such as pulmonary, nasal, buccal, rectal, sublingual, transdermal, and parenteral. As used herein, the term “parenteral” includes subcutaneous, intravenous, intra-arterial, intraperitoneal, intracardiac, intrathecal, and intramuscular injection, as well as infusion injections.

In instances where parenteral administration is utilized, it may be necessary to employ somewhat bigger oligomers than those described previously, with molecular weights ranging from about 500 to 30K Daltons (e.g., having molecular weights of about 500, 1000, 2000, 2500, 3000, 5000, 7500, 10000, 15000, 20000, 25000, 30000 or even more).

The method of administering may be used to treat any condition that can be remedied or prevented by administration of the particular conjugate. Those of ordinary skill in the art appreciate which conditions a specific conjugate can effectively treat. The actual dose to be administered will vary depend upon the age, weight, and general condition of the subject as well as the severity of the condition being treated, the judgment of the health care professional, and conjugate being administered. Therapeutically effective amounts are known to those skilled in the art and/or are described in the pertinent reference texts and literature. Generally, a therapeutically effective amount will range from about 0.001 mg to 1000 mg, preferably in doses from 0.01 mg/day to 750 mg/day, and more preferably in doses from 0.10 mg/day to 500 mg/day.

The unit dosage of any given conjugate (again, preferably provided as part of a pharmaceutical preparation) can be administered in a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

One advantage of administering the conjugates of the present invention is that a reduction in first pass metabolism may be achieved relative to the parent drug. Such a result is advantageous for many orally administered drugs that are substantially metabolized by passage through the gut. In this way, clearance of the conjugate can be modulated by selecting the oligomer molecular size, linkage, and position of covalent attachment providing the desired clearance properties. One of ordinary skill in the art can determine the ideal molecular size of the oligomer based upon the teachings herein. Preferred reductions in first pass metabolism for a conjugate as compared to the corresponding nonconjugated small drug molecule include: at least about 10%, at least about 20%, at least about 30; at least about 40; at least about 50%; at least about 60%, at least about 70%, at least about 80% and at least about 90%.

Thus, the invention provides a method for reducing the metabolism of an active agent. The method comprises the steps of: providing monodisperse or bimodal conjugates, each conjugate comprised of a moiety derived from a small molecule drug covalently attached by a stable linkage to a water-soluble oligomer, wherein said conjugate exhibits a reduced rate of metabolism as compared to the rate of metabolism of the small molecule drug not attached to the water-soluble oligomer; and administering the conjugate to a patient. Typically, administration is carried out via one type of administration selected from the group consisting of oral administration, transdermal administration, buccal administration, transmucosal administration, vaginal administration, rectal administration, parenteral administration, and pulmonary administration.

Although useful in reducing many types of metabolism (including both Phase I and Phase II metabolism) can be reduced, the conjugates are particularly useful when the small molecule drug is metabolized by a hepatic enzyme (e.g., one or more of the cytochrome P450 isoforms) and/or by one or more intestinal enzymes.

All articles, books, patents, patent publications and other publications referenced herein are incorporated by reference in their entireties. In the event of an inconsistency between the teachings of this specification and the art incorporated by reference, the meaning of the teachings in this specification shall prevail.

### Experimental

It is to be understood that while the invention has been described in conjunction with certain preferred and specific embodiments, the foregoing description as well as the examples that follow are intended to illustrate and not limit the scope of the invention. Other aspects, advantages and modifications within the scope of the invention will be apparent to those skilled in the art to which the invention pertains.

All non-PEG chemical reagents referred to in the appended examples are commercially available unless otherwise indicated. The preparation of PEG-mers is described in, for example, U.S. Patent Application Publication No. 2005/0136031.

¹H NMR (nuclear magnetic resonance) data was generated by a 300 MHz NMR spectrometer manufactured by Bruker. A list of certain compounds as well as the source of the compounds is provided below.

### Example 1

### mPEGₙ-Debrisoquin Synthesis

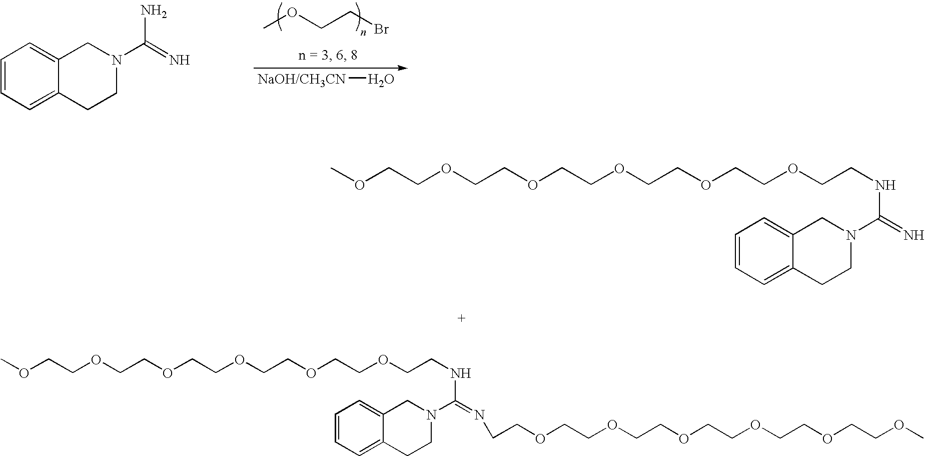

mPEGₙ-OMesylate: In a round bottom flask mPEGₙ-OH (n=3, 6, 8; 2 g) was added to triethylamine (1.7 mL) followed by the addition of dichloromethane (5 mL). The solution was placed in an ice bath and allowed to stir 30 minutes. Then methanesulfonyl chloride (1.08 mL) was added to the reaction flask and the reaction was allowed to stir overnight. Upon completion of the reaction, deionized water (15 mL) was added and the resulting mixture continued to stir for 30 minutes. The organic and aqueous layers were separated using a separatory funnel. The organic phase was washed with 0.1N HCl (1×100 mL) and water (1×100 mL), dried over sodium sulfate, filtered, and the solvent removed under reduced pressure.

mPEGₙ-Br: In a round bottom flask the above prepared mPEGₙ-Omesylate (n=3, 6, 8; 2.3 g) was added to tetrabutylammonium bromide (3.8 g) followed by the addition of acetonitrile (30 mL). The reaction solution was allowed to stir overnight at 50° C. under nitrogen. The solvent was removed under reduced pressure and the resulting liquid dissolved in an NaCl solution. The combined aqueous solution was extracted with ethyl acetate (3×50 mL), dried over sodium sulfate, filtered, and the solvent removed under reduced pressure.

mPEGₙ-N-Debrisoquin: In a pressurized reaction vessel, acetonitrile (8 mL) and debrisoquin were added. Then an NaOH solution (120 mg NaOH dissolved in 1 mL water) was added until the solution becomes clear. Finally, mPEGₙ-Br (250 mg) was added to the mixture and a nitrogen atmosphere is introduced into the pressurized vessel. The reaction is allowed to progress overnight at 70° C. and is monitored by analytical HPLC. Upon reaction completion, dichloromethane (200 mL) is added and the aqueous and organic layers separated using a separatory funnel. The organic phase was removed and the remaining aqueous phase was further extracted with dichloromethane (3×150 mL). The combined organic layers were dried over sodium sulfate and the solvent removed under reduced pressure. The resulting crude products were purified using Biotage Flash Chromatography (12+M, Water:CH₃CN, step gradient, flow rate: 12 mL/min). The final products were confirmed and characterized by NMR, HPLC, and MS.

### Example 2

### Analgesic Assay

An analgesic assay was used to determine whether a given compound can reduce and/or prevent visceral pain in mice.

The assay utilized CD-1 male mice (5-8 mice per group), each mouse being approximately 0.015-0.030 kg on the study day. Mice were treated according to standard protocols.

Mice were given a single “pretreatment” dose of a compound lacking covalent attachment of a water-soluble, non-peptidic oligomer, a corresponding version comprising the compound covalently attached to a water-soluble, non-peptidic oligomer, or control solution (IV, SC, IP or orally) thirty minutes prior to the administration of the acetic acid solution. The animal was given an IP injection of an irritant (acetic acid) that induces “writhing” which may include: contractions of the abdomen, twisting and turning of the trunk, arching of the back and the extension of the hindlimbs. Mice were given a single IP injection (0.1 mL/10 g bodyweight) of a 0.5% acetic acid solution. After the injection the animals were returned to their observation enclosure and their behavior was observed. Contractions were counted between 0 and 20 minutes after the injection. The animals were used once. Each tested article was dosed at 1, 3 and 10 mg/kg (n=5 animals/dose).

Table 1 below shows the analgesia activity of the tested compound against the standard analgesic, morphine.

TABLE 1

| | | | No. of | % Reduction | No. of | Responding |
|---|---|---|---|---|---|---|
| Compound | Mean | SEM | animals | in writhing | Responders | Fraction |
| mPEG8-N- | 33.6 | 8.5 | 5 | 33.2 | 2 | 0.40 |
| Debrisoquine | | | | | | |
| (10 mg/kg) | | | | | | |
| Debrisoquine | 49.6 | 4.5 | 5 | 10.6 | 0 | 0.00 |
| (10 mg/kg) | | | | | | |

### Example 3

### Radioligand Binding Assay for Debrisoquine

The binding affinities of debrisoquine sulfate (parent) and three mPEG conjugates are evaluated using radioligand binding assays in membranes prepared from rat forebrain which express noradrenalin transporters.

Competition binding experiments are conducted by incubating membranes with 1.0 nM of radioligand, [³H]-nisoxetine, in the presence of variable concentrations (0.1 nM to 3 μM and 1 nM to 30 μM for parent and PEG conjugates, respectively) of test compounds. The reaction is carried out in 50 mM Tris-HCl (pH 7.4), containing 300 mM NaCl and 5 mM KCl at 0-4° C. for 4 hours. Following incubations, the membranes are washed, and the bound radioactivity is measured. Non-specific binding is measured in the presence of excess desipramine (1.0 μM) as the cold ligand; this value is subtracted from the total binding to yield the specific binding at each test compound concentration.

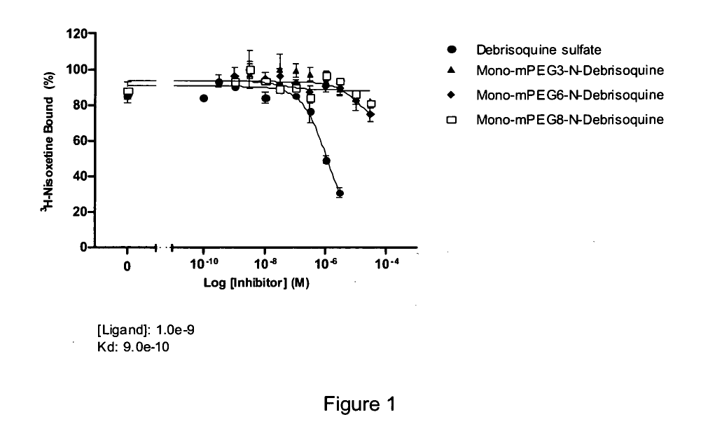

IC₅₀ values are obtained from non-linear regression analysis of dose-response curves (FIG. 1) and are calculated for those compounds that showed >50% inhibition of binding at the highest concentration tested. Kᵢ is obtained using the Cheng Prusoff correction using experimental K_{d} values that are previously determined under these assay conditions.

The binding affinity of parent debrisoquine to noradrenalin transporters is shown in Table 2. Since >50% inhibition of binding could not be observed for the mPEG conjugates at the highest test concentration of 30 μM, Kᵢ values could not be determined.

TABLE 2

| Test Compound | MW (Da) | Ki (μM) | Fold Change Relative to Parent |
|---|---|---|---|
| Debrisoquine | 224.3 | 0.594 | 1 |
| sulfate | | | |
| Mono-mPEG3-N- | 321.0 | — | — |
| Debrisoquine | | | |
| Mono-mPEG6-N- | 454.0 | — | — |
| Debrisoquine | | | |
| Mono-mPEG8-N- | 542.0 | — | — |
| Debrisoquine | | | |

1. A compound comprising a guanidine class compound residue covalently attached via a stable or degradable linkage to a water-soluble, non-peptidic oligomer.

2. The compound of claim 1, having the following structure: 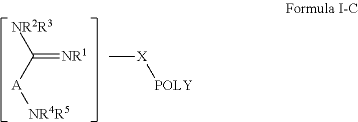
wherein:
A is selected from the group consisting of nil, lower alkylene, —CH₂NH—, and —CH₂CH₂NH—;
R¹ is selected from the group consisting of hydrogen, alkyl, cyano, and aryl;
R² and R³ are each independently selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl or heterocycle;
X is a spacer moiety; and
POLY is a water-soluble, non-peptidic oligomer.

3. The compound of claim 1 having the following structure: 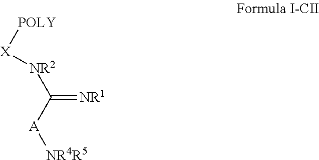
wherein:
A is selected from the group consisting of nil, lower alkylene, —CH₂NH—, and —CH₂CH₂NH—;
R¹ is selected from the group consisting of hydrogen, alkyl, cyano, and aryl;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl or heterocycle;
X is a spacer moiety; and
POLY is a water-soluble, non-peptidic oligomer.

4. The compound of claim 1 having the following structure: 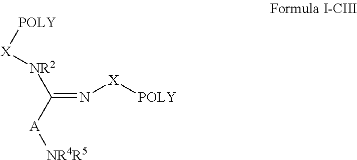
wherein:
A is selected from the group consisting of nil, lower alkylene, —CH₂NH—, and —CH₂CH₂NH—;
R² is selected from the group consisting of hydrogen, hydroxy, alkyl, substituted alkyl, aryl, substituted aryl, and alkylaryl;
R⁴ and R⁵ are each independently selected from the group consisting of hydrogen, alkyl, aryl, heteroaryl and alkylaryl, or R⁴, R⁵, and the nitrogen atom that they are attached to, together form heteroaryl or heterocycle;
each X is independently a spacer moiety; and
each POLY is independently a water-soluble, non-peptidic oligomer.

5. The compound of claim 1, wherein the guanidine class compound residue is a residue of a guanidine class compound selected from the group consisting of N-benzyl-N′,N″-dimethyl guanidine, N-2,4-dichlorobenzyl guanidine, N,N′-dimethyl-N″-3-methylbenzylguanidine, N-2-chlorobenzyl-N′,N″-dimethylguanidine, N,N′-dimethyl-N″-2-methylbenzyl guanidine, N-3-chlorobenzyl-N′,N″-dirnethylguanidine, N-2-chlorobenzyl-N′-methylguanidine, and N-ethyl-N′-methyl-N″-2-methylbenzylguanidine.

6. The compound of claim 1, wherein the guanidine class compound residue is a residue of a guanidine class compound having the following structure: 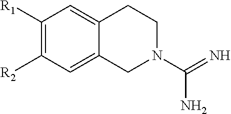
wherein:
R₁ and R₂ are individually selected from the group consisting of hydrogen, lower alkoxy, and taken together, lower alkylenedioxy.

7. The compound of claim 1, wherein the guanidine class compound residue is a residue of a guanidine class compound having the following structure: 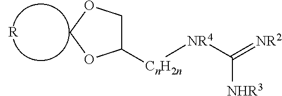
wherein:
n is an integer from 1-8; R², R³, and R⁴ each are members of the group consisting of hydrogen and lower alkyl; and R is an alkylene group containing 4 to 9 carbon atoms in the chain, with the proviso that when the alkylene group contains 4-5 carbon atoms, the chain can have fused thereto from 1 to 2 benzo rings, or the corresponding alkylene group can be substituted with up to 4 members of the group consisting of halo, lower alkyl, trifluoromethyl, trichloromethyl, lower alkoxy, benzyloxy, phenoxy, phenyl, hydroxy, carboxy, carbo-lower-alkoxy, nitro, sulfato and acetamido, said R group having a molecular weight less than 300.

8. The compound of claim 1, wherein the guanidine class compound residue is a residue of a guanidine class compound having the following structure: 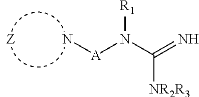
in which 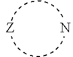
represents N,N-alkylene-imino, the alkylene portion of which contains from four to ten carbon atoms, A stands for lower alkylene containing from one to five carbon atoms, R₁ represents a member of the group consisting of hydrogen and lower alkyl, R₂ represents a member of the group consisting of hydrogen, lower alkyl, lower alkanoyl, benzoyl and methoxy-substituted benzoyl, and R₃ stands for a member of the group consisting of hydrogen and lower alkyl.

9. The compound of claim 1, wherein the guanidine class compound residue is a residue of a guanidine class compound having the following structure: 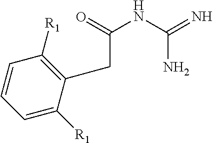
in which each R₁ is independently chloro or methyl.

10. The compound of claim 1, wherein the guanidine class compound residue is a residue of a guanidine class compound having the following structure: 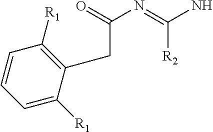
in which each R₁ is independently chloro or methyl and R₂ is lower alkoxy, lower alkylthio, or p-nitrobenzylthio.

11. The compound of claim 1, wherein the guanidine class compound residue is a residue of a guanidine class compound having the following structure: 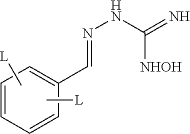
where each L, independently, represents hydrogen or halo having an atomic weight of about 19-36, provided at least one L is other than hydrogen.

12. The compound of claim 1, wherein the guanidine class compound residue is a residue of a guanidine class compound having the following structure: 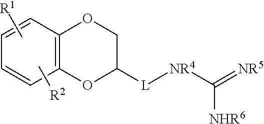
wherein: each R¹ and R² is independently selected from the group consisting of hydrogen, chloro, bromo, lower alkyl and lower alkoxy; L is selected from the group consisting of lower alkylene, —CH₂CH₂NH— and —CH₂NH—; R⁴ is selected from the group consisting of hydrogen and lower alkyl; and R⁵ and R⁶, when taken together with the nitrogen atoms to which they are attached and the carbon atom which is geminal to said nitrogen atoms, are imidazolinyl.

13. The compound of claim 1, wherein the guanidine class compound residue is a residue of a guanidine class compound having the following structure: 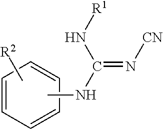
in which the R¹-substituted cyano-guanidyl radical is placed in the 2-, 3-, or 4-position of the pyridine ring, and in which R¹ stands for aliphatic hydrocarbon having from 1 to 8 carbon atoms, cycloalkyl having from 3 to 8 carbon atoms, phenyl, benzyl, or phenethyl, and R² stands for hydrogen, halo, hydroxyl, lower alkyl or lower alkoxy radicals.

14. The compound of claim 1, wherein the guanidine class compound residue is a residue of a guanidine class compound is selected from the group consisting of bethanidine, debrisoquin, guanabenz, guanadrel, guanathidine, guanafacine, guanoxabenz, guanoxan, and pinacidil.

15. The compound of claim 1, wherein the water-soluble, non-peptidic oligomer is a poly(alkylene oxide).

16. The compound of claim 15, wherein the poly(alkylene oxide) is a poly(ethylene oxide).

17. The compound of claim 1, wherein the water-soluble, non-peptidic oligomer has between 1 and 30 monomers.

18. The compound of claim 17, wherein the water-soluble, non-peptidic oligomer has between 1 and 10 monomers.

19. The compound of claim 16, wherein the poly(alkylene oxide) includes an alkoxy or hydroxy end-capping moiety.

20. The compound of claim 1, wherein a single water-soluble, non-peptidic oligomer is attached to the guanidine class compound residue.

21. The compound of claim 1, wherein more than one water-soluble, non-peptidic oligomer is attached to the guanidine class compound residue.

22. The compound of claim 1, wherein the guanidine class compound residue is covalently attached via a stable linkage.

23. The compound of claim 1, wherein the guanidine class compound residue is covalently attached via a degradable linkage.

24. The compound of claim 1, wherein the linkage is an ether linkage.

25. The compound of claim 1, wherein the linkage is an ester linkage.

26. A composition comprising a compound comprising a guanidine class compound residue covalently attached via a stable or degradable linkage to a water-soluble, non-peptidic oligomer, and optionally, a pharmaceutically acceptable excipient.

27. A composition of matter comprising a compound comprising a guanidine class compound residue covalently attached via a stable or degradable linkage to a water-soluble, non-peptidic oligomer, wherein the compound is present in a dosage form.

28. A method comprising covalently attaching a water-soluble, non-peptidic oligomer to a guanidine class compound.

29. A method of treatment comprising administering a compound comprising a guanidine class compound residue covalently attached via a stable or degradable linkage to a water-soluble, non-peptidic oligomer to a subject in need thereof.
